(19) United States
(12) Patent Application Publication
(10) Pub. No.: US 2026/0198857 A1
(43) Pub. Date: Jul. 16, 2026

(54) MEASUREMENT ARRANGEMENT OF WEARABLE TRAINING COMPUTER

(71) Applicant: Polar Electro Oy, Kempele (FI)

(72) Inventors: Lauri Lumme, Kempele (FI); Olli Komulainen, Kempele (FI)

(73) Assignee: Polar Electro Oy, Kempele (FI)

(21) Appl. No.: 19/135,394

(22) PCT Filed: Dec. 7, 2023
(86) PCT No.: PCT/FI2023/050669

§ 371 Date: Jun. 4, 2025

(30) Foreign Application Priority Data
Dec. 8, 2022 (GB) 2218435.2

(51) Int. Cl. A61B 5/00; A61B 5/256; A61B 5/282; A61B 5/332; A61B 90/00

(52) U.S. Cl. CPC A61B 5/681; A61B 5/256; A61B 5/282; A61B 5/332; A61B 5/7285; A61B 2090/065; A61B 2562/182

(57) ABSTRACT

A wearable training computer includes a casing having lugs for attaching a wristband to the casing, wherein at least one lug includes at least one electrode coupled with a biometric measurement circuitry arranged in the casing, and a processing circuitry configured to control the biometric measurement circuitry to perform an electric measurement based on a skin contact with at least one lug including the electrode, and further to compute at least one bioparameter on the basis of measurement data received from the biometric measurement circuitry and to output the at least one heart activity parameter to a user of the wearable training computer.

### CROSS-REFERENCE TO RELATED APPLICATIONS

This application is a national phase application of International Application No. PCT/FI2023/050669, filed Dec. 7, 2023, which claims benefit and priority to Great Britain Application No. 2218435.2, filed Dec. 8, 2022, which are incorporated by reference herein in their entireties.

### BACKGROUND

### Technical Field

The invention relates to a field of wearable training computers, especially an electrocardiography measurement arrangement of the wearable training computers.

### SUMMARY

Electrocardiography measurement arrangements are widely used in the wearable training computers for measuring an electrocardiogram. Bioimpedance is another parameter that has been incorporated into wearable training computers. Both measurements are based on the use of electrodes and skin contact. The known measurement arrangements in the field of the wearable training computers have drawbacks especially from ergonomics point of view causing challenges to the measurement. The aim of the invention is to alleviate these drawbacks.

The present invention is defined by the subject matter of the independent claim.

Embodiments are defined in the dependent claims.

The embodiments and features, if any, described in this specification that do not fall under the scope of the independent claim are to be interpreted as examples useful for understanding various embodiments of the invention.

### BRIEF DESCRIPTION OF THE DRAWINGS

In the following the invention will be described in greater detail by means of preferred embodiments with reference to the attached drawings, in which

FIG. 1 illustrates a wearable training computer according to an embodiment of the invention;

FIGS. 2, 4 and 5 illustrate a casing of the wearable training computer from different angles according to an embodiment of the invention; and

FIGS. 3A and 3B illustrate using of the wearable training computer according to an embodiment of the invention.

### DETAILED DESCRIPTION

The following embodiments are exemplifying. Although the specification may refer to “an”, “one”, or “some” embodiment(s) in several locations of the text, this does not necessarily mean that each reference is made to the same embodiment(s), or that a particular feature only applies to a single embodiment Single features of different embodiments may also be combined to provide other embodiments.

Embodiments of the invention relate to a wearable training computer configured to carry out measurements during a physical exercise performed by a user. The wearable training computer may be a portable system attachable to the user's body. The wearable training computer is configured to measure physiological training data from the user's performance during the physical exercise and to output the training data to the user via a user interface of the training computer and/or via a user interface of another apparatus.

In an embodiment, the wearable training computer may further comprise an apparatus configured to be attached to an object. Such an apparatus may comprise an attachment structure designed and arranged to receive the training computer in a fixed, integrated, or detachable manner and to attach the training computer to the object. The attachment may be realized by a band that may be designed to encircle the object such that the band is attached around the object. The band may comprise locking parts at ends of the band where the locking parts form mutually counterparts such as a buckle and a catch. The locking parts may fix the band around the object as is commonly known in the field of wristwatches, wrist computers etc. The object may be the user and the band may be designed to be attached around the user's wrist, making the wearable training computer a wrist device. Other forms of attachment of wearable devices are equally possible, e.g. the training computer may be integrated or attached to a garment such as a shirt, pants, harness, strap, or headwear.

The wearable training computer may be used for measuring an electric bioparameter on the used. An example of the bioparameter is electrocardiogram (ECG). The ECG may be used for measuring heart activity, e.g. a heart rate, a respiratory rate, or various cardiac parameters extractable from QRS waveforms comprised in the ECG. Alternatively, the ECG may be used as a reference for other heart activity measurements such as pulse transit time (PTT) measured by using the ECG and photoplethysmogram (PPG) measured on the user's wrist, for example. One or more electrocardiogram electrodes (ECG sensors) are used for measuring the ECG. Another example of the electric bioparameter is bioimpedance that may be used, for example, for measuring body composition. Muscle and blood containing a high amount of water has low resistivity (impedance) while fat has high resistivity.

In the field of the wearable training computers, the electrodes are often placed some specific part of a casing of the wearable training computer on which one or more fingers are placed to measure the ECG or bioimpedance, for example. In other words, there are one or more measuring points in the casing configured to receive a skin contact of the finger(s) of the user of the wearable training computer. A size of the casing of the wearable training computer is relatively small causing challenges to ergonomics. Conventionally, at least two electrodes are required, and the electrodes should be disposed such that one electrode contacts one hand and the other would contact the other hand of the user. However, there exist ECG implementations where only one electrode is needed for skin contact. One electrode may be on a bottom surface of the casing that faces the skin, when the wearable training computer is worn. However, the remaining space of the casing is often occupied by the display and by the buttons. Due to the small size of the casing, reading of a screen of the wearable training computer may be difficult when the fingers are placed on the measuring points, if the electrode were placed on the front surface of the casing. In other words, the fingers, when placed on the measuring points may block or limit visibility of the screen. Furthermore, the fingers may have to be placed to spots that are not ergonomically comfortable. The invention is aimed to alleviate the issues of the know solutions.

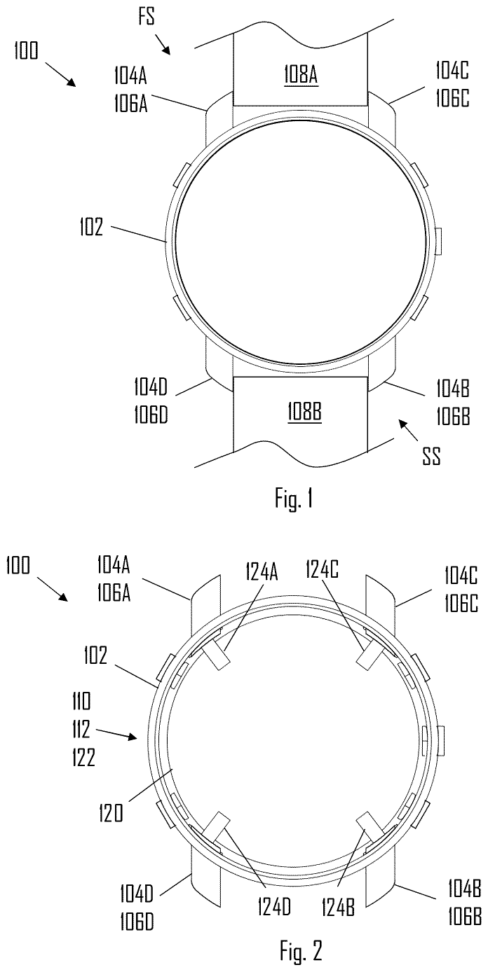

Referring to FIG. 1, according to an aspect, there is provided a wearable training computer 100 comprising a casing 102 having lugs 104A-D for attaching a wristband 108A-B to the casing 102, wherein at least one lug 104A-D comprises at least one electrode 106A-D coupled with a biometric measurement circuitry 110 arranged in the casing 102, and a processing circuitry 112 configured to control the biometric measurement circuitry 110 to perform an biometric measurement based on a skin contact with at least one lug 104A-D comprising the electrode 106A-D, and further to compute at least one bioparameter on the basis of biometric measurement data received from the biometric measurement circuitry 110 and to output the at least one bioparameter to a user of the wearable training computer 100.

FIG. 1 illustrates the wearable training computer 100 with a first and a second end of the wristband 108A-B coupled with the casing. The wristband is used to wear the wearable training computer to a wrist of the user. The casing 102 comprises totally four lugs 104A-D for attaching the ends of the wristband 108A-B to the casing 102. The lug is a projection in the casing acting as an attaching mechanism. The wearable training computer is illustrated in FIG. 1 in a position in which it is normally used in the wrist of the user, for example. A first side FS (upside) of the casing 102 comprises two lugs 104A, 104C for attaching the first end of the wristband 108A, and a second side SS (underside) of the casing 102 comprises two lugs 104B, 104D for attaching the second end of the wristband 108B. The end of the wristband is set between the lugs and attached with them by a pin, for example. The pin is coupled with the end of the wristband. A first end of the pin is attached with the one lug and a second end of the pin is attached with another lug on the same side (upside or downside). For example, one pin is set between the lugs 104A and 104C for attaching the first end of the wristband 108A, and another pin is set between the lugs 104B, 104D for attaching the second end of the wristband 108B as illustrated in FIG. 1. Each lug may comprise a feature like a hole for receiving the one end of the pin. The pin itself is not visible in FIG. 1.

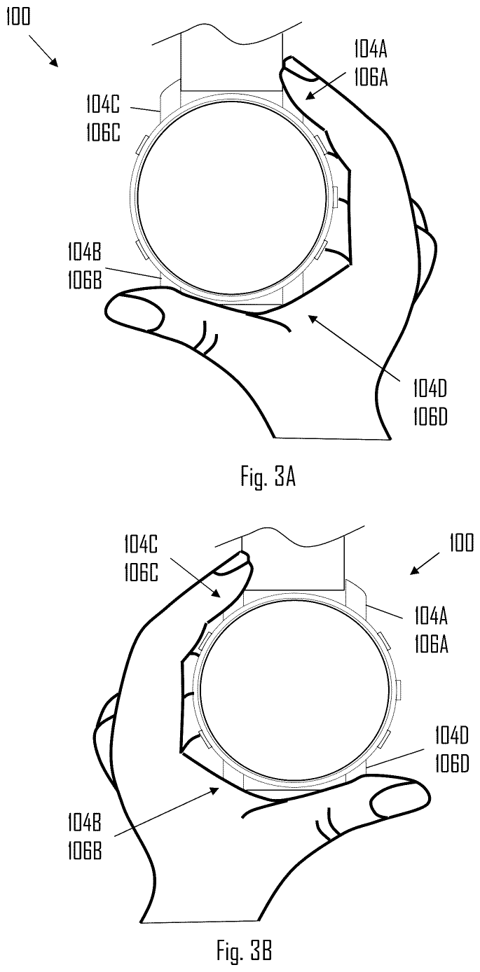

At least one lug 104A-D comprises the at least one electrode 106A-D coupled with a (biometric) measurement circuitry 110) arranged in the casing 102 wherein the electrode and the biometric measurement circuitry are configured to be used for the measurement of the bioparameter. FIG. 2 illustrates the wearable training computer 100 in an open state such that a printer circuit board (PCB) 120 inside the casing 102 is visible. The measurement circuitry 110 may be assembled on the PCB 120 having a connection to the one or more electrodes 106A-D arranged outside of the casing. The PCB may further comprise the processing circuitry 112 configured to control the measurement circuitry 110 to perform the measurement. The measurement may be performed based on the skin contact with the one or more lugs comprising the electrode. In other words, the user of the wearable training computer may touch the at least one lug having the electrode for example by the finger and the skin contact of the finger may be used for measuring the ECG.

The processing circuitry 112 is further configured to compute the at least one bioparameter based on measurement data received from the measurement circuitry 110 and to output the at least one bioparameter to the user of the wearable training computer 100. The at least one bioparameter may comprise a bioparameter based on the ECG and/or bioimpedance, e.g. heart rate, body composition, respiratory rate, heart stroke volume, blood pressure, or (de)hydration status. The at least one bioparameter may be presented to the user via user interface of the wearable training computer. The user interface may refer to a (touch) screen of the wearable training computer, for example. The bioparameter(s) may also be presented in a web-based application, for example.

The bioparameter may be an electric bioparameter, as described herein. Since the lug is a mechanical feature protruding from the casing, touching of the lug is easy improving the ergonomics of the ECG-measurement. Furthermore, touching of the lug does not limit the visibility of the screen of the user interface. Therefore, a technical effect of the invention is improved ergonomics of the ECG- and/or bioimpedance measurement and better visibility of the screen during the measurement.

Still referring to FIGS. 1 and 2, in an embodiment, the wearable training computer 100 comprises a first lug 104A comprising a first electrode 106A and a second lug 104B comprising a second electrode 106B, wherein the biometric measurement circuitry 110 is configured to perform the measurement based on the skin contact with at least the first and the second lugs 104A, 104B. In this embodiment, the measurement is performed based on the skin contact of two fingers of the user. One finger is set on the first lug comprising the electrode, and another finger is set on the second lug, for example.

In an embodiment, one of the first and the second lugs (comprising the electrode) is configured to provide a measurement signal, and another one is for grounding. Both lugs may, however, be coupled to inputs of the measurement circuitry for producing the measurement data. For example, the first lug may provide the ECG signal and the second lug may be for grounding, or the other way around. Both first and second lug may then be coupled to respective inputs of a differential amplifier of the biometric measurement circuitry.

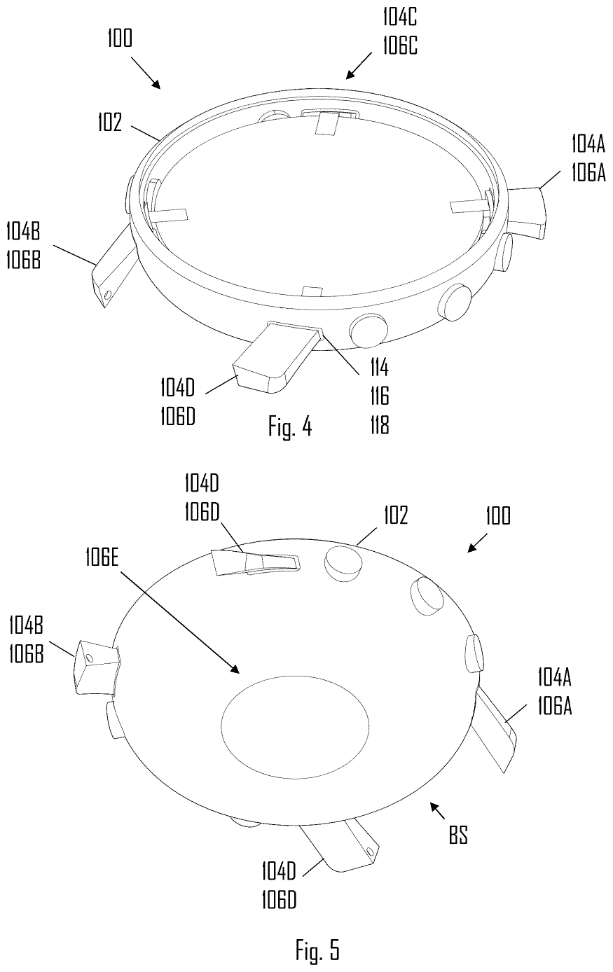

Still referring to FIGS. 1 and 2, in an embodiment, the first lug 104A comprising the first electrode 106A and the second lug 104B comprising the second electrode 106B are on the opposite sides of the casing 102. In a first example, the opposite side may refer to an arrangement in which one of the lugs 104A, 104B is on the first side FS of the casing 102, and another one of the lugs 104A, 104B is on the second side SS of the casing 102. For example, as illustrated in FIGS. 2 and 3, the first lug 104A with the first electrode 106A is on the first side FS of the casing 102 and the second lug 104B with the second electrode 106B is on the second side SS of the casing 102. In a second example, the opposite side may refer to an arrangement in which the first and the second lugs are on the same side (the first or the second side of the casing). Then the first lug may be on the left side and the second lug may be on the right side of the first side of the casing, for example. In other words, both lugs (first and second) may be on the upside or downside of the casing (from the perspective of the appended Figures).

Referring now to FIG. 3A, in an embodiment, the first lug 104A with the first electrode 106A is on the right-side of the first side FS of the casing 102, and the second lug 104B with the second electrode 106B is on the left side of the second side SS of the casing 102. It is also possible that the first lug is on the right side of the first side, and the second lug is on the left side of the second side. In this embodiment, the opposite side means that the lugs are on the opposite sides of the casing (upside/downside) and further on the different side of the opposite sides (left/right).

Still referring to FIG. 3A in which the first lug 104A and electrode 106A are on the right side of the first side FS of the casing 102 and the second lug 104B and electrode 106B are on the left side of the second side SS of the casing 102.

The user of the wearable training computer may set, when performing the measurement, a thumb on the second lug and a forefinger on the first lug as illustrated in FIG. 3A. This is suitable finger position especially for persons that wear the wearable training computer around the left wrist. The screen is between the fingers, and they do not limit the visibility of the screen, and further the ergonomics is pleasant (natural). In other words, there is no needs to set fingers any unpleasant and complex positions which may block the visibility of the screen of the wearable training computer.

Referring to FIGS. 1 and 2, in an embodiment, the wearable training computer 100 further comprises a third lug 104C comprising a third electrode 106C, and a fourth lug 104D comprising a fourth electrode 106D, wherein the biometric measurement circuitry 110 is configured to perform the measurement based on the skin contact with at least the third and the fourth lugs 104C, 104D. Hence, the casing may comprise totally four lugs wherein all the lugs comprise respective electrodes for measuring the bioparameter(s).

In an embodiment, one of the third and the fourth lugs comprising the electrode is configured to provide the measurement signal, and another one of the third and the fourth lugs is for grounding. Both lugs may, however, be coupled to inputs of the measurement circuitry for producing the measurement data. For example, the third lug may provide the ECG-signal and the fourth lug may be for grounding, or the other way around.

Still referring to FIGS. 1 and 2, in an embodiment, the third lug 104C comprising the third electrode 106C and the fourth lug 104D comprising the fourth electrode 106D are on the opposite sides of the casing 102. In other words, the third and the fourth lugs may be arranged on the casing the same way as the first and the second lugs described above. The term “opposite” in the case of the third and the fourth lugs may refer to the same arrangements as described above with the first and the second lugs.

In an embodiment, illustrated in FIGS. 1 and 2, the first and the third lugs 104A, 104C are on the first side FS of the casing 102 such that the first lug 104A is on the right side and the third lug 104C is on the left side of the first side FS. Hence, the first and the third lugs 104A, 104C are configured to be used to attach the first end of the wristband 108A. The second and the fourth lugs 104B, 104D are on the second side SS of the casing 102 such that the second lug 104B is on the left side and the fourth lug 104D is on the right side of the second side FS. Hence, the second and the fourth lugs 104B, 104D are configured to be used to attach the second end of the wristband 108B.

Referring now to FIG. 3B, in an embodiment, the third lug 104C and the electrode 106C are on the left side of the first side FS of the casing 102, and the fourth lug 104D and the electrode 106D are on the right side of the second side SS of the casing 102. The user of the wearable training computer may set, when performing the ECG-measurement, the thumb on the fourth lug and the forefinger on the third lug as illustrated in FIG. 3B. This is suitable finger position especially for persons wearing the wearable training computer around the right-hand wrist. Then the fingers do not limit the visibility of the screen and further the ergonomics is pleasant (natural). In other words, there is no needs to set fingers any unpleasant and complex positions which may block the visibility of the screen of the wearable training computer.

In an embodiment, the casing comprises a total of four lugs with the respective electrodes wherein any of them alone or any combination of them can be used for measuring the bioparameter. Hence, the user may select the preferred lug or lugs and he/she can use it for the measurements. As described above, the combination of the opposite first and second lugs may be used by persons wearing the apparatus in the left hand, and the combination of the opposite third and fourth lugs may be used by the persons wearing the apparatus in the right hand, for example.

Referring to FIG. 2, in an embodiment, each of said at least one lug 104A-D is made of electrically conducting material, thus forming the respective electrode 106A-106D. The conductive material may comprise metal, for example. Hence, the conductive lug acts as an electrode such that the measurement may be performed based on the skin contact with at least one conductive lug without any separate electrodes such as a coating. The at least one lug 104A-D may comprise a conductor 124A-D configured to couple the at least one lug 104A-D with the measurement circuitry 110. In another embodiment, the lug(s) comprising the electrode(s) is coated with electrically conductive coating. The lug(s) may still be of electrically conductive material and form a part of the signal path(s) from the electrode(s) to the measurement circuitry. In embodiments where multiple lugs are coated with the electrically conductive coating and form a part of the same measurement arrangement, the coating may be identical or substantially electrical in the multiple lugs. This enables similar electrical characteristics to the electrodes, thus improving the measurement performance.

Referring now to FIG. 5, in an embodiment, the wearable training computer 100 further comprises at least one electrode 106E arranged on a bottom surface BS of the wearable training computer 100 that faces the skin of the user when the wearable training computer is worn, wherein the measurement circuitry 110 is configured to perform the measurement based on the skin contact with the at least one electrode arranged on the bottom and at least one lug 104A-D comprising the electrode 106A-D. The wearable training computer may comprise a conductive part, coupled with the measurement circuitry by a conductor, at the bottom surface that may act as an electrode. The conductive part is set against the skin of the user when the wearable training computer is worn.

FIG. 3A illustrates that the measurements may be performed based on the skin contact on the first and the second electrodes. In addition to the first the second electrodes, the electrode on the bottom surface facing against the skin of the user may be used for measuring. Then the bioparameter may be measured based on three electrodes in which the skin of the forefinger may against the first lug, the skin of the forefinger may be against the second lug and the electrode at the bottom surface may be against the skin of a wrist of the user. Hence, there may be three measurement points wherein two of the points is for providing the measurement signal and one point is for grounding. For example, one of the lugs is for grounding, and another lug and the electrode at the bottom provides the measurement signals. The same principles are valid also in the situation of FIG. 3B in which the third and the fourth lugs are used instead of the first and the second lugs.

Referring now to FIG. 4, an interface between the casing 102 and the at least one of the lugs 104A-104C with the electrode 106A-106D comprises an insulator 114 configured to electrically isolate the lug 106A-106D from the casing 102. The interface refers to a connection area in which the lug is coupled with the casing. The purpose of the isolation is to avoid disturbances during the measurements and to improve a quality of the measurement results. The insulator may be made of non-conductive material like plastics, for example. The insulator may comprise a hole for the conductor coupling the at least one lug to the measurement circuitry arranged inside the casing to enable the measurements based on the skin contact with the lug such that the lug is isolated form the casing.

Still referring to FIG. 4, in an embodiment, the interface comprises an opening 116 in the casing 102 configured to receive the lug 104A-104D, wherein the insulator 114 is arranged at least partly between inner surfaces of the opening 116 and surfaces of the lug 104A-104D that are inside the opening 116. Hence, the lug may, at least partly, go into the hole of the casing such that there is the insulator between them electrically isolating the lug from the casing. In an embodiment, the insulator may further be used for attaching non-conductively the at least one lug to the casing.

In an embodiment, the interface further comprises an ingress protection element (IP-element) 118 for sealing the interface. The IP-element may prevent ingress of dust and water inside the casing, in other words, it makes the interface waterproof. In an embodiment, the insulator 114 may comprise the IP-element 118.

Referring still to FIG. 4, in an embodiment, a surface of the lug 104A-104D that is configured to receive the skin contact is, at least partly, flat. The skin contact may take place on a top surface of the lug. The top surface may refer to a surface which is on the top when the wearable training computer is worn according to the normal use (see FIGS. 3A-B). The flat surface enables the bigger contact area between the skin and the lug enabling the better contact. Alternatively, the surface may be curved or a combination of curved and flat surfaces. The surface may be polished, or it may be mechanically textured. Mechanical texturing may refer to milling or nibbling the metal surface of the lug(s), thus providing a roughened surface that may improve the skin contact.

In an embodiment, the wearable training computer 100 further comprises a sensing element 122, coupled with the processing circuitry 112, configured to detect the skin contact on the at least one lug 104A-D comprising the electrode 106A-D, wherein upon detecting the skin contact, the sensing element is configured to provide a control signal to the processing circuitry 112 to activate the biometric measurement circuitry 110 to perform the measurements. In other words, the sensing element is configured to detect the skin contact on the lug, and when the skin contract is detected, it provides the control signal to the processing circuitry to activate the measurement circuitry for measuring the bioparameter. Hence, the measurements are automatically activated when the skin contact exists, and separate activation by the user of the wearable training computer may be avoided. For example, if the first and the second lugs are used for measuring the ECG, the activation may take place when the sensing element has detected the skin contact in both lugs. The skin contact may be based on measuring impedance between the first and second lugs (and in some embodiments between the third and fourth lugs). Without the skin contact, the lugs are isolated from one another, and the skin contact electrically couples the lugs together. So when the impedance is detected to be in a certain range indicating the skin contact, the measurements may be triggered.

In an embodiment, the sensing element 122 is configured to provide the control signal to the processing circuitry 112 to activate the measurement circuitry 110 when the skin contact exists for a predetermined time. In other words, there may be a time limit for the skin contact before the activation takes place. Then unnecessary activations may be avoided if the skin contact exists accidentally (quickly) on the lugs. The time limit may be a value between 2-5 seconds, for example.

In an embodiment, the processing circuitry is configured to disable a signal path of the third and fourth lugs 104C-104D when the measurement is performed based on the first and second lugs 104A-104B, and respectively to disable the signal path of the first and second lugs 104A-104B when the measurement is performed based on the third and fourth lugs 104C-104D. FIG. 3A illustrates the situation in which the bioparameter is measured based on the skin contact of the first and the second lugs and the signal bath from the third and the fourth lugs, on which the skin contact does not exist, may be disabled. FIG. 3B illustrates the situation in which the bioparameter is measured based on the skin contact of the third and the fourth lugs and the signal bath from the first and the second lugs, on which the skin contact does not exist, may be disabled. Disabling of the unused signal bath (first/second or third/fourth) reduces noise improving the results of the measurement.

In an embodiment, the lugs 104A-D comprise a hole configured to receive a locking member for attaching the wristband 108A-B wherein the hole and/or the locking member comprises an insulator configured to electrically isolate the locking member from the lug 104A-D. The locking member may be the pin as described above. The insulator is configured to electrically isolate the pin and the lug(s), and also isolate two adjacent lugs from each other in which the pin is assembled. Referring to FIG. 1, the pin may be used to attach the first and the second ends of the wristbands 108A-B to the casing 102. The pin may extend between the lugs that are on the same side of the casing (first/second side). The isolation ensures that these pins are not electrically coupled with the lugs improving the quality of the measurements.

The lug(s) coupled to the measurement circuitry also enable coupling the band electrically to the measurement circuitry. In an embodiment, the wearable training computer further comprises the band comprising at least one electric component, and at least one signal path coupling the at least one electric component to the measurement circuitry and/or the processing circuitry via at least one of the lugs. The at least one electric component may comprise a power generator such as a wearable solar panel or panels, and the at least one signal path comprises a power conductor. The at least one electric component may comprise a body temperature sensor, and the at least one signal path comprises a measurement signal path for a body temperature signal. The at least one signal path may be arranged via a lug comprising the at least one electrode or via a lug not comprising an electrode. In the embodiment where a signal path is arranged via the lug comprising the electrode, the signal path from the band to the measurement circuitry and the signal path from the electrode of the lug to the measurement circuitry may be separated galvanically, or they may utilize the same galvanic contact. In the latter case, the measurement circuitry or the processing circuitry may comprise at least one switch controlling which one of the at least one electric component of the band and the lug electrode is coupled to the measurement circuitry at a time. The other one will be isolated by the switch at the time. Other means for multiplexing the two signal paths to the same galvanic contact (signal line) may be used.

The invention provides a structure for measuring the bioparameter in the wearable training computers that reduces many drawbacks of the known solutions. The visibility of the screen of the wearable training computer is improved when the measurement points are on the lugs since the lugs extends from the casing and therefore the finger(s) used in the measurement are placed further away from the casing. If the fingers are set directly against the casing, they often block the visibility of the screen at least partly. Furthermore, the ergonomics of the measuring is optimal when the measurement points are placed in the opposite lugs. When the casing comprises four measurement points, both the left-and right-handed users may find the optimal position of the fingers as illustrated in FIGS. 3A and 3B.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), graphics processing units (GPUs), processors, controllers, micro-controllers, microprocessors, other electronic units designed to perform the functions described herein, or a combination thereof. For firmware or software, the implementation can be carried out through modules of at least one chipset (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

It will be obvious to a person skilled in the art that, as the technology advances, the inventive concept can be implemented in various ways. The invention and its embodiments are not limited to the examples described above but may vary within the scope of the claims.

What is claimed is:

1. A wearable training computer comprising:
a casing having lugs configured to attach a wristband to the casing;
wherein at least one lug comprises at least one electrode coupled with a biometric measurement circuitry arranged in the casing; and
a processing circuitry configured to control the biometric measurement circuitry to perform an electric measurement based on a skin contact with at least one lug comprising the electrode, and further to compute at least one electric bioparameter on the basis of measurement data received from the measurement circuitry and to output the at least one electric bioparameter to a user of the wearable training computer.

2. The wearable training computer of claim 1, wherein the wearable training computer comprises a first lug comprising a first electrode and a second lug comprising a second electrode, the biometric measurement circuitry being configured to perform the electric measurement based on the skin contact with at least the first and the second lugs.

3. The wearable training computer of claim 2, wherein one of the first and the second lugs is configured to provide a measurement signal, and another one of the first and the second lugs is configured to provide grounding.

4. The wearable training computer of claim 2, wherein the first lug and the second lug are on opposite sides of the casing.

5. The wearable training computer of claim 2, wherein the wearable training computer further comprises a third lug comprising a third electrode, and a fourth lug comprising a fourth electrode, the biometric measurement circuitry being configured to perform the electric measurement based on the skin contact with at least the third and the fourth lugs.

6. The wearable training computer of claim 5, wherein one of the third and the fourth lugs is configured to provide the measurement signal, and another one of the first and the second lugs is configured to provide grounding.

7. The wearable training computer of claim 5, wherein the third lug and the fourth lug are on opposite sides of the casing.

8. The wearable training computer of claim 5, wherein the processing circuitry is configured to disable a signal path of the third and fourth lugs when the electric measurement is performed based on the first and second lugs, and to disable a signal path of the first and second lugs when the electric measurement is performed based on the third and fourth lugs.

9. The wearable training computer of claim 1, wherein each of the at least one lug is made of electrically conducting material, thereby forming the respective electrode.

10. The wearable training computer of claim 1, wherein the wearable training computer further comprises at least one electrode arranged on a bottom surface of the wearable training computer that faces the skin when the wearable training computer is worn and is coupled with the biometric measurement circuitry, the biometric measurement circuitry being configured to perform the electric measurement based on the skin contact with the at least one electrode arranged on the bottom and at least one lug comprising the electrode.

11. The wearable training computer of claim 1, wherein an interface between the casing and the at least one of the lugs comprising the electrode, comprises an insulator configured to electrically isolate the lug from the casing.

12. The wearable training computer of claim 11, wherein the interface comprises an opening in the casing configured to receive the lug, the insulator being arranged at least partly between inner surfaces of the opening and surfaces of the lug that are inside the opening.

13. The wearable training computer of claim 11, wherein the interface further comprises an ingress protection element configured to seal the interface.

14. The wearable training computer of claim 1, wherein the wearable training computer further comprises a sensing element, coupled with the processing circuitry, configured to detect the skin contact on the at least one lug comprising the electrode, upon detecting the skin contact, the sensing element being configured to provide a control signal to the processing circuitry to activate the biometric measurement circuitry to perform the electric measurement.

15. The wearable training computer of claim 14, wherein the sensing element is configured to provide the control signal to the processing circuitry to activate the biometric measurement circuitry when the skin contact exists for a predetermined time.

16. The wearable training computer of claim 1, wherein the lugs comprise a hole configured to receive a locking member configured to attach the wristband, the hole and/or the locking member comprising an insulator configured to electrically isolate the locking member from the lug.

17. The wearable training computer of claim 1, further comprising:
a band comprising at least one electric component; and
at least one signal path coupling the at least one electric component to the measurement circuitry and/or the processing circuitry via at least one of the lugs.

18. The wearable training computer of claim 17, wherein the at least one signal path is arranged via a lug comprising the at least one electrode.

19. The wearable training computer of claim 6, wherein the third lug and the fourth lug are on opposite sides of the casing.

20. The wearable training computer of claim 12, wherein the interface further comprises an ingress protection element configured to seal the interface.
